(19) United States
(12) Patent Application Publication
(10) Pub. No.: US 2026/0315683 A1
(43) Pub. Date: Oct. 15, 2026

(54) POSITIVE-NEGATIVE PRESSURE NASAL IRRIGATOR

(71) Applicant: Mainiusi Intelligent Medical Technology (Suzhou) Co., Ltd., Suzhou (CN)

(72) Inventors: Chengcheng Cao, Suzhou (CN); Tao Sun, Suzhou (CN)

(21) Appl. No.: 19/646,389

(22) Filed: Apr. 13, 2026

(30) Foreign Application Priority Data
Apr. 14, 2025 (CN) 202510461346.X
Apr. 14, 2025 (CN) 202520695749.6
Apr. 14, 2025 (CN) 202520699181.5
Apr. 18, 2025 (CN) 202520741809.3
Oct. 27, 2025 (CN) 202522266380.X

(51) Int. Cl. A61M 3/02

(52) U.S. Cl. CPC A61M 3/0258; A61M 3/0202; A61M 3/0245; A61M 2205/8206; A61M 2210/0618

(57) ABSTRACT

A positive-negative pressure nasal irrigator comprises a vacuum pump, wherein a positive pressure output port of the vacuum pump is in communication with a clean solution chamber, and a negative pressure output port of the vacuum pump is in communication with a waste solution chamber; the vacuum pump compresses gas into the clean solution chamber to apply positive pressure thereto, pushing the rinsing solution through the first liquid tube to the solution outlet to enter the nasal cavity in the form of liquid flow or atomized spray; subsequently, the negative pressure output port of the vacuum pump sucks gas through the second air tube to generate negative pressure in the waste solution chamber, so that after flowing out of the nasal cavity, the rinsing solution flows back into the waste solution chamber under the action of negative suction and natural gravity.

### TECHNICAL FIELD

The invention relates to the technical field of nasal irrigators, in particular to a positive-negative pressure nasal irrigator.

### BACKGROUND ART

A nasal irrigator is a device for rinsing the nasal cavity. It typically delivers rinsing solution into the nostrils under a certain pressure (suction, gravity, or mechanical pressure), allowing the solution to flow through the nasal vestibule (the exposed part of the nose), paranasal sinuses, and nasal passages, bypassing the nasopharynx, and then drain out from the other nostril or the mouth. Existing nasal irrigators, such as the one disclosed in Chinese Patent No. CN106265042B, mainly include a water pump and a battery. This design has certain drawbacks in practical use. Since it relies on only one water pump to generate positive pressure inside the nasal cavity to drive fluid flow, users who need nasal irrigation usually suffer from nasal congestion or blockage. With only positive pressure applied internally, the rinsing solution may fail to flow out normally from the opposite nostril. Under forced positive pressure, the solution may even flow into other areas, which not only prevents smooth nasal irrigation but also causes severe discomfort.

### SUMMARY OF THE INVENTION

In view of the above background, the invention provides a positive-negative pressure nasal irrigator, wherein a vacuum pump compresses gas to apply positive pressure to a clean solution chamber, pushing the rinsing solution from a liquid inlet toward a solution outlet. After circulating through the entire nasal cavity, the used solution is recovered into a negative-pressure waste solution chamber via a waste solution return port. The combined action of positive pressure, negative suction, and natural gravity improves the flow efficiency of the rinsing solution in the nasal cavity, enabling rapid nasal irrigation, preventing solution retention or blockage in the nasal cavity, achieving smooth nasal rinsing, and enhancing comfort.

The technical solution of the invention is as follows:
a positive-negative pressure nasal irrigator, comprising:
an irrigation head, a body of the irrigation head is provided with a solution outlet and a waste solution return port corresponding to two nostrils;
a liquid chamber assembly located below the irrigation head, including a clean solution chamber and a waste solution chamber, wherein the clean solution chamber is in communication with the solution outlet, and the waste solution chamber is in communication with the waste solution return port;
a vacuum pump, wherein a positive pressure output port of the vacuum pump is in communication with the clean solution chamber, and a negative pressure output port of the vacuum pump is in communication with the waste solution chamber.

Further, the vacuum pump is connected to a positive pressure regulating tube, the positive pressure output port on the positive pressure regulating tube communicates with the clean solution chamber via a first air tube, a first liquid tube is arranged inside the clean solution chamber, and one or more liquid inlets are provided at a lower end of the first liquid tube, a positive pressure relief port on the positive pressure regulating tube is connected to a positive pressure relief tube.

Further, the irrigation head is provided with a liquid outlet chamber corresponding to the solution outlet, and an upper section of the first liquid tube is located inside the liquid outlet chamber.

Further, the vacuum pump is connected to a negative pressure regulating tube, the negative pressure output port on the negative pressure regulating tube communicates with the waste solution chamber via a second air tube; an upper outlet end of the second air tube is located at an upper part of the waste solution chamber, a negative pressure relief port on the negative pressure regulating tube is connected to a negative pressure relief tube.

Further, the positive pressure relief tube and the negative pressure relief tube are open to the atmosphere.

Further, the second air tube is arranged close to an inner wall of the waste solution chamber.

Further, an atomizing head is provided at a port of the first liquid tube.

Further, the nasal irrigator further comprises a main unit including a main housing, wherein the vacuum pump is disposed inside the main housing. A battery and a circuit board are also arranged inside the main housing, and the circuit board is electrically connected to the battery and the vacuum pump.

Further, control buttons are arranged on the circuit board, and the control buttons are provided with keys.

The beneficial effects of adopting the above technical solution are as follows: the invention uses a vacuum pump to compress gas into the clean solution chamber to apply positive pressure, pushing the rinsing solution through the first liquid tube to the solution outlet, where it enters the nasal cavity either as a liquid flow or as atomized droplets via the atomizing head. After circulating through the nasal cavity, the solution is drawn into the negative-pressure waste solution chamber from the waste solution return port under negative suction and natural gravity. This improves the flow efficiency of the rinsing solution in the nasal cavity, enables rapid irrigation, prevents solution blockage in the nasal cavity, ensures smooth nasal rinsing, and enhances comfort.

In addition, the negative-pressure waste solution chamber avoids the risk of rinsing solution entering other areas due to excessive positive pressure, thereby preventing discomfort and making the irrigation process gentler, safer, and more comfortable.

The air passages and liquid passages of the invention are independent of each other, and the rinsing solution does not circulate through the vacuum pump body, ensuring that the rinsing solution is not contaminated.

### BRIEF DESCRIPTION OF ACCOMPANY DRAWINGS

To more clearly illustrate the embodiments of the invention or the technical solutions in the prior art, the accompanying drawings used in the description of the embodiments or the prior art will be briefly introduced below.

FIG. 1 is a structural view of Embodiment 1 of the invention.

FIG. 2 is an exploded structural view of the main unit in Embodiment 1 of the invention.

FIG. 3 is a first sectional structural view of Embodiment 1 of the invention.

FIG. 4 is a second sectional structural view of Embodiment 1 of the invention.

FIG. 5 is a third sectional structural view of Embodiment 1 of the invention.

FIG. 6 is a sectional structural view of Embodiment 2 of the invention.

FIG. 7 is a structural view of the vacuum pump in an embodiment of the invention.

FIG. 8 is a structural view of the liquid chamber assembly in an embodiment of the invention.

FIG. 9 is a top view of the main unit in an embodiment of the invention.

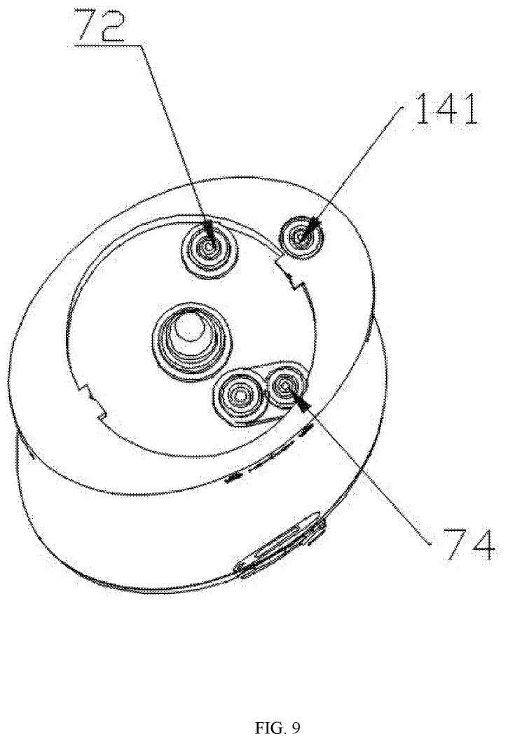

### SPECIFIC EMBODIMENT OF THE INVENTION

To make the objectives, technical solutions, and advantages of the embodiments of the invention clearer, the technical solutions in the embodiments of the invention will be clearly and completely described below with reference to the accompanying drawings in the embodiments of the invention.

The invention will be further described below with reference to the accompanying drawings.

### Embodiment 1

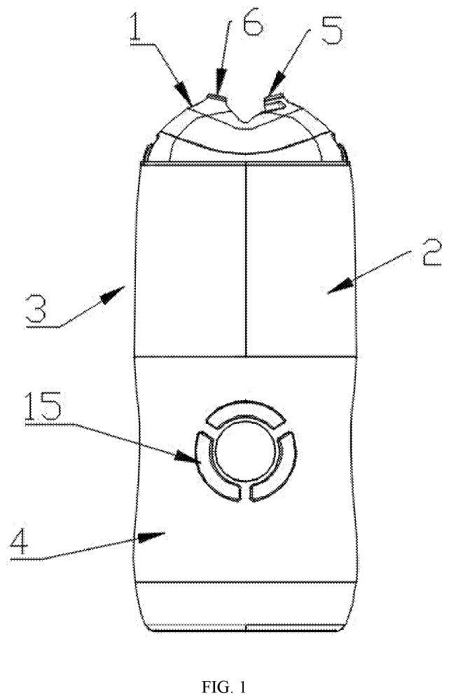

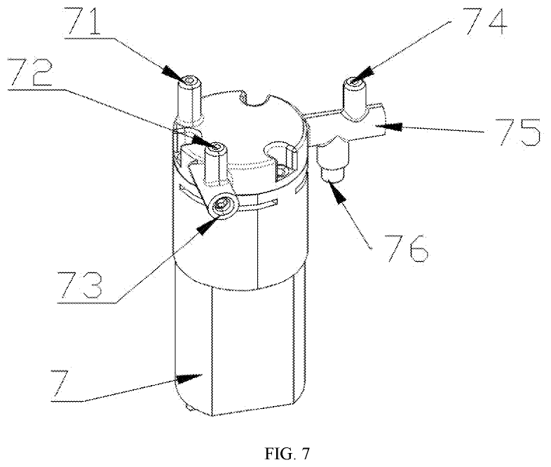

As shown in FIG. 1 to FIG. 5 and FIG. 7 to FIG. 8, a positive-negative pressure nasal irrigator, comprising:
an irrigation head 1, a body of the irrigation head 1 is provided with a solution outlet 5 and a waste solution return port 6 corresponding to the two nostrils;
a liquid chamber assembly located below the irrigation head 1, including a clean solution chamber 2 and a waste solution chamber 3, wherein the clean solution chamber 2 is in communication with the solution outlet 5, and the waste solution chamber 3 is in communication with the waste solution return port 6;
a vacuum pump 7, wherein a positive pressure output port 76 of the vacuum pump is in communication with the clean solution chamber 2, and a negative pressure output port 71 of the vacuum pump is in communication with the waste solution chamber 3.

The vacuum pump 7 compresses gas into the clean solution chamber 2 through the positive pressure output port 76 to create positive pressure therein, thereby driving the rinsing solution in the clean solution chamber 2 to the solution outlet 5. The vacuum pump 7 sucks and exhausts gas from the waste solution chamber through the negative pressure output port 71, so that the used rinsing solution flows back into the waste solution chamber under negative suction and natural gravity.

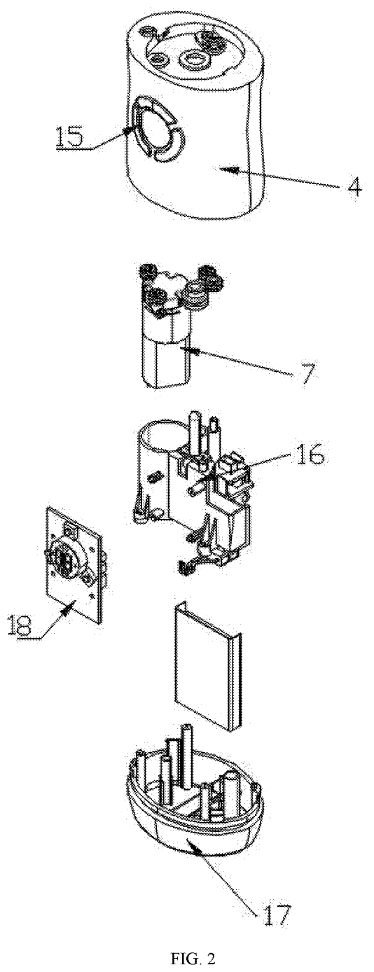

As shown in FIG. 2, the positive-negative pressure nasal irrigator further comprises a main unit 4 including a main housing, wherein the vacuum pump 7 is disposed inside the main housing. A battery and a circuit board 18 are also arranged inside the main housing, and the circuit board 18 is electrically connected to the battery and the vacuum pump 7. Control buttons are arranged on the circuit board 18, and the control buttons are provided with keys 15. Further, an inner housing 16 for fixing the vacuum pump is arranged inside the main housing, and a bottom shell 17 is provided at a bottom of the main housing. The bottom shell can be detachably connected to the main housing via a snap-fit structure or integrally formed with the main housing.

In an alternative embodiment, the clean solution chamber and the waste solution chamber may be arranged vertically. Specifically, the clean solution chamber and the waste solution chamber may be arranged either side by side or vertically, and the main unit is shaped accordingly to match the layout.

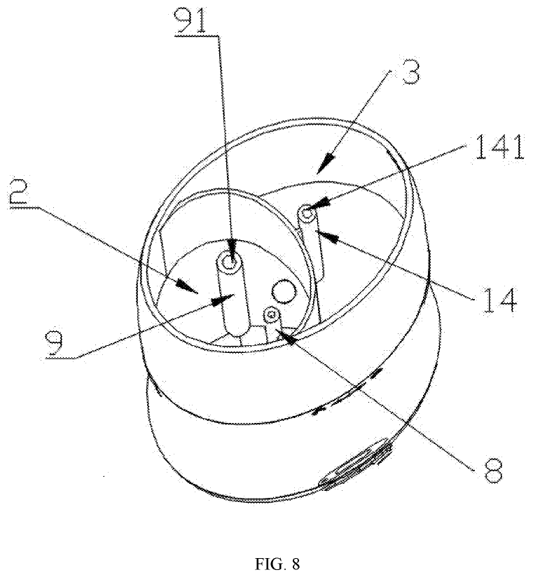

In this embodiment, as shown in the figures, the clean solution chamber and the waste solution chamber are arranged side by side. Specifically, as shown in FIG. 8, the liquid chamber assembly comprises a liquid chamber housing located below the irrigation head. The liquid chamber housing is divided by a middle partition into a clean solution chamber (positive pressure chamber) 2 and a waste solution chamber (negative pressure chamber) 3. The clean solution chamber and the waste solution chamber may be of separate design or integrated design. The liquid chamber assembly is located above the main unit, and the liquid chamber housing may be detachably connected to the main housing via a snap-fit structure or integrally formed with the main housing.

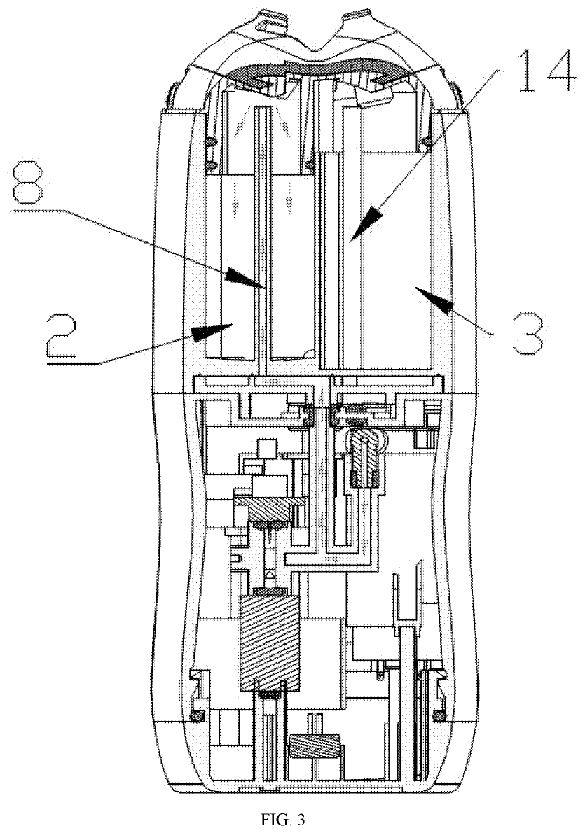

Specifically, as shown in FIG. 3 to FIG. 5 and FIG. 7, the vacuum pump 7 is connected to a positive pressure regulating tube 75. The positive pressure output port 76 on the positive pressure regulating tube 75 communicates with the clean solution chamber 2 via a first air tube 8. A first liquid tube 9 is arranged inside the clean solution chamber 2, and one or more liquid inlets 10 are provided at a lower end of the first liquid tube 9. A positive pressure relief port 74 on the positive pressure regulating tube 75 is connected to a positive pressure relief tube. The irrigation head 1 is provided with a liquid outlet chamber 11 corresponding to the solution outlet 5, and a liquid outlet 91 at an upper section of the first liquid tube 9 is located inside the liquid outlet chamber 11, thereby preventing the rinsing solution from affecting the first air tube 8.

The vacuum pump 7 is connected to a negative pressure regulating tube. The negative pressure output port 71 on the negative pressure regulating tube communicates with the waste solution chamber 3 via a second air tube 14; an air port 141 at an upper end of the second air tube 14 is located at an upper part of the waste solution chamber 3. A negative pressure relief port 72 on the negative pressure regulating tube is connected to a negative pressure relief tube.

The positive pressure relief tube and the negative pressure relief tube are open to the atmosphere to release excess pressure. The positive pressure relief tube and the negative pressure relief tube are respectively connected to relief ports, which are not limited to the vacuum pump and its pipelines, and may also be provided on the irrigation head, the clean solution chamber, or the waste solution chamber. Control valves are respectively arranged on the positive pressure relief tube and the negative pressure relief tube to control the relief operation.

A port of the positive pressure regulating tube 75 serves as a positive pressure regulating port, and a port of the negative pressure regulating tube serves as a negative pressure regulating port 73. The positive pressure regulating port and the negative pressure regulating port allow stepless pressure regulation. The positive pressure output port and the negative pressure output port respectively provide power to the positive pressure chamber and the negative pressure chamber. Thus, a single vacuum pump can achieve both positive pressure irrigation and negative pressure suction, and the positive irrigation pressure and negative suction pressure can be adjusted according to user needs to achieve a better cleaning effect.

The second air tube 14 is arranged close to an inner wall of the waste solution chamber 3.

A dust cover is fitted on the irrigation head 1. The irrigation head is integrally made of silica gel to ensure structural stability and provide a more comfortable contact with the skin.

In another embodiment, sealing structures, such as interference-fit gaskets or sealing rings, are provided between air passages, liquid passages, and an interior of the main unit. Sealing structures, such as interference-fit gaskets or sealing rings, are also provided at all joints of the main housing for improved waterproof performance.

### Embodiment 2

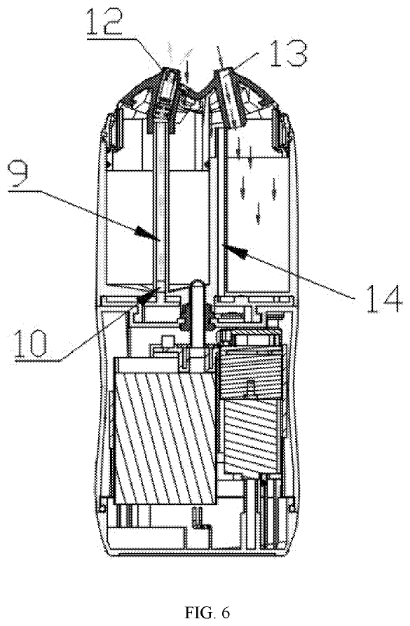

As shown in FIG. 6, compared with Embodiment 1, this embodiment is provided with an atomizing head 12 at a port of the first liquid tube 9. The irrigation head with the atomizing head 12 at the port of the first liquid tube 9 is a dual-nozzle spray irrigation and suction head, from which the rinsing solution is sprayed in mist form through the solution outlet 5.

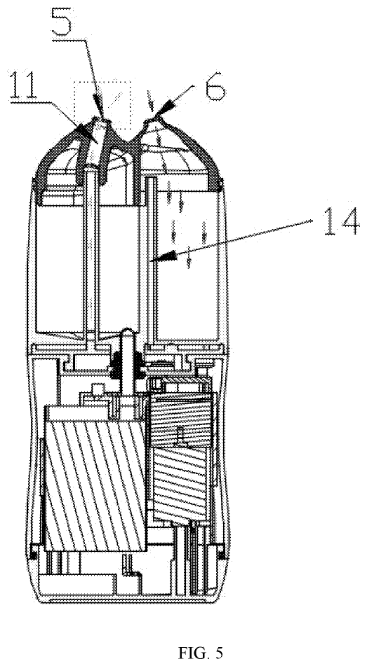

As shown in FIG. 5, Embodiment 1 is not provided with an atomizing head 12 at the port of the first liquid tube 9, forming a dual-nozzle liquid-flow irrigation and suction head, from which the rinsing solution is ejected in liquid flow form through the solution outlet 5.

As shown in FIG. 6, the irrigation head 1 is provided with a liquid return chamber 13 corresponding to the waste solution return port 6. An upper port of the second air tube 14 extends beyond a lower opening of the liquid return chamber 13, so that waste solution does not flow directly into the air tube but preferentially sinks to a bottom of the waste solution chamber for collection.

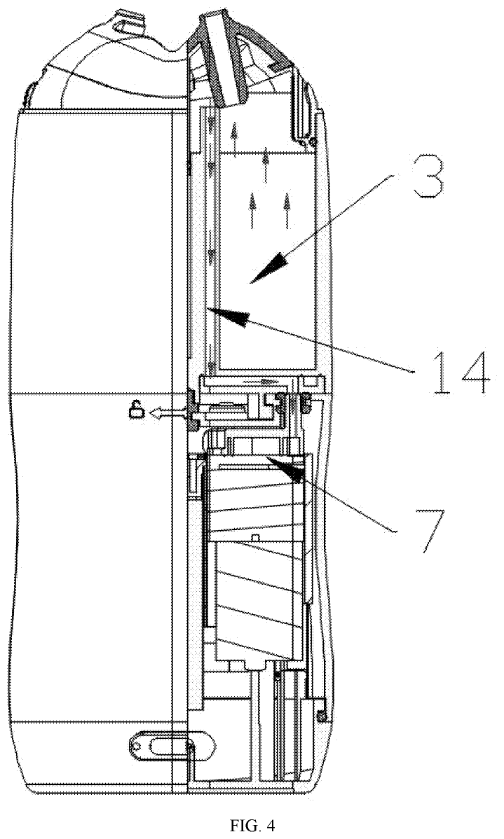

As shown in FIG. 3 to FIG. 6, arrows in FIG. 3 indicate the flow direction of positive pressure air; the arrows in FIG. 4 indicate the flow direction of negative pressure air; the left arrows in FIG. 5 and FIG. 6 indicate the flow direction of the rinsing solution, and the right arrows indicate the flow direction of the waste solution. It can be seen that the air passages and liquid passages are independent of each other, and liquid is transmitted only through the liquid tubes without circulating through the vacuum pump body. Thus, neither the rinsing solution nor the waste solution passes through concealed parts, eliminating cleaning dead zones. Preferably, the first air tube and the first liquid tube are detachably connected to the vacuum pump and the clean solution chamber, and the second air tube and the second liquid tube are detachably connected to the vacuum pump and the waste solution chamber, facilitating thorough cleaning and ensuring hygienic use.

In the invention, the vacuum pump compresses gas into the clean solution chamber. When the clean solution chamber is under positive pressure, it pushes the rinsing solution from the liquid inlet to the solution outlet, where the solution enters the nasal cavity as a liquid flow or atomized droplets via the atomizing head. The waste solution return port is in communication with the atmosphere or the nasal cavity, while other parts are sealed. The negative pressure output port of the vacuum pump sucks gas through the second air tube to create negative pressure in the waste solution chamber. After circulating through the entire nasal cavity, the rinsing solution is drawn into the negative-pressure waste solution chamber from the waste solution return port under negative suction and natural gravity, allowing the solution to flow smoothly from one nostril to the other or to the mouth, significantly improving irrigation efficiency. The invention uses only one vacuum pump to simultaneously generate positive pressure and negative pressure, and completely separates the air and liquid passages, so that the rinsing solution and waste solution do not pass through any motor or concealed pipelines.

The above describes the invention and its embodiments, and such description is non-limiting. The structures shown in the accompanying drawings are only one of the embodiments of the invention, and the actual structure is not limited thereto. In summary, any person skilled in the art, inspired thereby, may devise structures and embodiments similar to this technical solution without departing from the inventive concept and without creative effort, and such structures and embodiments shall fall within the protection scope of the invention.

1. A positive-negative pressure nasal irrigator, comprising:
an irrigation head, a body of the irrigation head is provided with a solution outlet and a waste solution return port corresponding to two nostrils;
a liquid chamber assembly located below the irrigation head, including a clean solution chamber and a waste solution chamber, wherein the clean solution chamber is in communication with the solution outlet, and the waste solution chamber is in communication with the waste solution return port;
a vacuum pump, wherein a positive pressure output port of the vacuum pump is in communication with the clean solution chamber, and a negative pressure output port of the vacuum pump is in communication with the waste solution chamber.

2. The positive-negative pressure nasal irrigator of claim 1, wherein the vacuum pump is connected to a positive pressure regulating tube, the positive pressure output port on the positive pressure regulating tube communicates with the clean solution chamber via a first air tube, a first liquid tube is arranged inside the clean solution chamber, and one or more liquid inlets are provided at a lower end of the first liquid tube, a positive pressure relief port on the positive pressure regulating tube is connected to a positive pressure relief tube.

3. The positive-negative pressure nasal irrigator of claim 2, wherein the irrigation head is provided with a liquid outlet chamber corresponding to the solution outlet, and an upper section of the first liquid tube is located inside the liquid outlet chamber.

4. The positive-negative pressure nasal irrigator of claim 2, wherein the vacuum pump is connected to a negative pressure regulating tube, the negative pressure output port on the negative pressure regulating tube communicates with the waste solution chamber via a second air tube; an upper outlet end of the second air tube is located at an upper part of the waste solution chamber, a negative pressure relief port on the negative pressure regulating tube is connected to a negative pressure relief tube.

5. The positive-negative pressure nasal irrigator of claim 4, wherein the positive pressure relief tube and the negative pressure relief tube are open to atmosphere.

6. The positive-negative pressure nasal irrigator of claim 4, wherein the second air tube is arranged close to an inner wall of the waste solution chamber.

7. The positive-negative pressure nasal irrigator of claim 2, wherein an atomizing head is provided at a port of the first liquid tube.

8. The positive-negative pressure nasal irrigator of claim 1, wherein the nasal irrigator further comprises a main unit including a main housing, wherein the vacuum pump is disposed inside the main housing; a battery and a circuit board are also arranged inside the main housing, and the circuit board is electrically connected to the battery and the vacuum pump.

9. The positive-negative pressure nasal irrigator of claim 8, wherein control buttons are arranged on the circuit board, and the control buttons are provided with keys.
